(19) United States
(12) Patent Application Publication
(10) Pub. No.: US 2014/0303082 A1
(43) Pub. Date: Oct. 9, 2014

(54) SELECTIVE GPCR LIGANDS

(71) Applicant: Medical Research Council, Swindson Wiltshire (GB)

(72) Inventor: Andreas Tzakos, Loannina (GR)

(21) Appl. No.: 14/310,016

(22) Filed: Jun. 20, 2014

Related U.S. Application Data
(63) Continuation of application No. PCT/EP2012/005323, filed on Dec. 21, 2012.

(30) Foreign Application Priority Data
Dec. 23, 2011 (GB) 1122261.9
Mar. 27, 2012 (GB) 1205397.1

(51) Int. Cl. C07K 7/14; C07K 7/06; G06F 19/16; C07K 5/08

(52) U.S. Cl. CPC C07K 7/14; C07K 5/08; C07K 7/06; G06F 19/16
USPC 514/9.7; 530/331; 530/328; 530/316; 703/11

(57) ABSTRACT

In the past decade a great deal of structural information for class A-GPCRs (G protein-coupled receptors) has emerged. However, the structural and electronic basis of ligand selectivity for closely related receptor subtypes such as the angiotensin receptors AT1aR and AT2R, which present completely diverse biological functions in response to the same ligand, is poorly understood. In order to monitor complex responses in biosystems it is useful to have ligands that present a gradient in terms of selectivity. In this study we present an efficient method to tune ligand selectivity for the two angiotensin II receptor subtypes, AT1aR and AT2R, by controlling aromatic-prolyl interactions in angiotensin II, through alternation of aromatic electronics. On the basis of this strategy, an AT2R selective and high affinity agonist analogue (Ki=3 nM) was obtained.

### CROSS-REFERENCE TO RELATED APPLICATIONS

This application is a continuation of International Application No. PCT/EP2012/005323, filed on 21 Dec. 2012, which claims priority to GB1205397.1, filed on 27 Mar. 2012, and to GB1122261.9, filed on 23 Dec. 2011, which applications are all hereby incorporated by reference in their entirety.

### INTRODUCTION

GPCRs are key determinants of signal transduction from the extracellular milieu to the intracellular space^{1, 2}. Although they can be activated by an array of extracellular ligands ranging from small neurotransmitters to hormones, the sequence conservation in key structural elements of rhodopsin-like GPCRs³ propose a common activation mechanism. The recent X-ray structures of GPCRs^{1, 4-7} defined the overall architecture of the GPCRs-family A and pin-pointed the structure of the ligand-binding pocket. However, these structures also raised questions about the mechanism of ligand selectivity to closely related receptor subtypes^{1, 8}. For instance, even though residues that directly surround the ligand binding pocket of human b1 and b2 adrenoreceptors appear to be identical, ligands bind with completely different specificities the two receptor subtypes^{1, 8}.

Therefore, in GPCRs molecular recognition is not a trivial process dependant solely on the receptor molecular architecture, but is strongly associated with the ligand structure. This is evident by the mechanism of rhodopsin activation, where the transition to its signaling state is accomplished by a cis-to-trans isomeric structural switch of its intrinsic ligand retinal^{9, 10}. The major consequence of this cis/trans switch is to break and/or weaken most of the electrostatic restraints between the transmembrane helices triggering receptor activation¹⁰. Such cis-to-trans isomerization switch is emerging as a critical component of numerous biological processes, with proline being a key player^{11, 14}.

The bioactive hormone angiotensin II (AII: DRVYIHPF) has a proline residue in its primary structure whose isomeric state could be of importance in the activation of its AT1a and AT2 receptor subtypes. In aqueous solution the prevalent conformer of the native AII is the trans (>95%)¹⁵. Interestingly, the Pro7Gly mutation in AII that provides conformational plasticity, almost retained its affinity for the AT2R, whereas presented 150 times lower affinity for AT1aR¹⁶.

A key question that emerges is whether a reduction in the energy barrier of the cis/trans interconversion would allow AII to simultaneously populate two different conformations (cis and trans), with consequences on the binding affinity and specificity for the two AII receptor subtypes. The putative role of proline isomerization in receptor subtype selectivity could be most valuable, since the effects of the AT2R activation (vasodilation, apoptosis and antiproliferation) oppose those mediated by AT1aR (cellular growth and proliferation for AT1)¹⁷⁻¹⁹. In addition, since AT2R activation suppresses the growth of pancreatic carcinoma cells, this receptor is a potential target of chemotherapy against this type of cancer^{20, 21}. Therefore, a fine tuning of the different functional responses of AT1a and AT2 receptors by a combinatorial use of regulatory ligands could be a powerful therapeutic tool²².

Here, we describe a novel strategy to fine-tune ligand selectivity for the AT1aR and AT2R subtypes through electronic control of ligands aromatic-prolyl interactions. A novel low nM affinity and selective agonist for AT2R was established on the basis of this strategy.

### SUMMARY OF THE INVENTION

In a first aspect of the present invention, there is provided a process for preparing a selective ligand for an angiotensin II receptor comprising the steps of:

(i) selecting a motif of a ligand for the receptor which is susceptible to cis-trans isomerisation;

(ii) comparing the ligand with sequences in a database comprising the motif and determining the influence of substitutions in or close to this motif on the formation of a cis or trans isomer;

(iii) effecting a substitution in the ligand in accordance with the comparison in step (ii), thus favouring a cis or trans isomer in the ligand.

Development of selective ligands for closely related G protein-coupled receptor subtypes, although important to decode receptor pathophysiological responses, is a tedious and time consuming process. In the absence of a comprehension of the interaction determinants of ligand binding and subtype selectivity, trial and error and serendipity play a major role in the successful development of selective ligands. Governing the principles for achieving selective ligand recognition is especially important when receptor subtypes present opposite functions. This is clearly evident for the two AII receptor subtypes:

AT1aR has been implicated in numerous pathologies, such as heart failure, atherosclerosis, retinopathy, cardiac hypertrophy, vascular smooth muscle proliferation, and hypertension, to name some³⁷. On the contrary, AT2R mediate rather different functions to those of AT1R, such as antiproliferation, antiinflammation, neuronal differentiation, vascular remodeling and tumor suppression^{20, 21, 38-40}. AT2R has therefore been assigned as an important pharmaceutical drug target. Due to the absence of detailed knowledge of ligand-receptor recognition interactions, the identification of selective ligands for AT2R came after long and delicate efforts⁴¹.

We have developed a strategy to tune ligand-receptor selectivity for the two receptors subtypes using ligands that are recognized with similar affinity by the hormone AII.

In order for a polypeptide to exhibit cis-trans isomerisation, an N-substituted amino acid is required. Examples include sarcosine and praline. Preferably, in the process according to claim 1, the motif comprises proline. The sequence Pro-X, where X is any amino acid, endows the polypeptide with similar energies for both cis and trans isomers; this means that both isomers are theoretically possible. The nature of X affects the equilibrium between the cis and trans isomers.

In a preferred embodiment, there is provided a process according to the invention wherein the motif is X₁-Pro-X₂, wherein X₁ and X₂ are the same or different and can be any amino acid. X₁ also has an effect on the balance between cis and trans isomers. By selecting suitable amino acids at each of these positions, ligands can be tailored to favour one or the other isomeric form, or in some instances to be capable of occupying both isomeric forms.

In one embodiment of the invention, X₂ is Phe. Thus, ligands comprising the motif X-Pro-Phe can be analysed for the capacity to favour cis or trans isomeric forms; the prevalence of cis or trans isomers can be evaluated in a database such as pdb, and a likelihood of formation of such an isomer assigned to the test ligand on the basis of the identity of X in the X-Pro-Phe motif.

By such a method, a ligand can be designed which is selective for AT2R, or AT1R. For example a ligand which is selective for AT2R can display increased cis-isomerisation in solution.

A source of motifs for use in the method of the invention, and therefore as basis for potential ligands, are the various forms of angiotensin. For example, the ligand can be a mutant of Angiotensin I, II, III or IV, or saralasin. Saralasin is a derivative of Angiotensin II in which the N- and C-terminal amino acids are substituted with Sarcosine and Alanine respectively.

In one embodiment, the motif is the His⁶-Pro⁷-Phe⁸ motif in Angiotensin II (AII).

In one embodiment, the His⁶ residue is replaced with Tyr, creating a Tyr-Pro-Phe motif, and a Tyr⁶ analogue of AII. Accordingly, in a second aspect, there is provided a ligand for the Angiotensin II receptor having the sequence Asp-Arg-Val-Tyr-Ile-Tyr-Pro-Phe.

In an alternative embodiment, a 4-substituted Phe residue in which an electron-donating or an electron-withdrawing group is introduced at this position is used in position 6 of AII. Accordingly, there is provided a ligand for the Angiotensin II receptor having the sequence Asp-Arg-Val-Tyr-Ile-Phe-Pro-Phe wherein Phe⁶ is substituted at the 4 position, i.e. substituting the hydrogen in the para-position of the phenylalanine ring.

The ligands are, in one embodiment, selective for the AT2 receptor. In an alternative embodiment, the ligand is selective for the AT1 receptor. In general, electron-donating substitutions at position 6 favour selectivity for AT2R, and electron-withdrawing substitutions favour selectivity for AT1R.

The ligand AII receptor subtype selectivity can be precisely sculpted by tuning the electronic character of a simple substitution of the hydrogen in the para-position of phenylalanine introduced at position 6 of AII (4-x-Phe⁶). Specifically, the [Y]⁶-AII analogue with an electron-donating group (—OH) resulted in a selective and high affinity binder for AT2R (Ki=3.4±0.8 nM), whereas electron-withdrawing groups completely abolished high binding affinity and selectivity for this receptor (FIG. 5). Most importantly, this receptor recognition phenotype is directly correlated to the architecture of the cis character and the compactness of the 4-x-Phe⁶-Pro⁷-Phe⁸ motif induced by this electronic control. AII analogues containing electron-deficient aromatic residues at position 6 relatively disfavoured cis amide bonds and presented reduced selectivity and affinity for the AT2 receptor in contrast to electron-rich aromatic residues. For instance, [4-NO₂—F]⁶-AII displayed 300 times lower affinity for AT2R in comparison to [Y]⁶-AII, but high affinity (nM) for AT1aR (unpublished data). In the same line, [F]⁶-AII presented the same affinity for both AT1aR and AT2R but more than 50 times reduced affinity in comparison to the affinity of [Y]⁶-AII for AT2R (unpublished data). This cis-trans isomerization control is based on the tuning of the interactions between Pro⁷ and aromatic ring electronics of 4-x-Phe⁶.²⁷ Thus, the cis form is stabilized through a CH-π interaction developed among the electron deficient prolyl C—H bonds and electron-rich aromatic ring⁴². Indeed, our NMR data indicated in the [Y]⁶-AII AT2 selective analogue both an enhancement of the cis character and a ring packing of the two aromatic side-chains around the proline (Y⁶—P⁷—F⁸, FIG. 2a). This residue packing results in a protection of the implicated peptide bonds, as determined by amide proton temperature coefficient studies and diffusion ordered experiments (FIG. 2c,d), thus, lowering the cost of transferring them into the more hydrophobic environment of the AT2R ligand-binding pocket.

This is the first time that a strategy is described to control ligand receptor subtype selectivity via delicate tuning of aromatic electronics. The selective and high affinity AT2R analogue, [Y]⁶-AII, derived in the frame of this strategy, stimulates the activity of AT2R in PC12 cells (FIG. 4).

In a further aspect, there is provided a ligand selected according to the foregoing aspect of the invention for use in tumour therapy.

It is known that AT2R is a target for tumour therapy. In particular, it has been shown that AT2R knockout promotes pancreatic tumour progression²⁰, and overexpression of AT2R induces cell death in lung adenocarcinoma²¹. Accordingly, the ligands according to the present invention, which selectively activate the AT2R, are candidates for anti-tumour therapy.

Preferably, the ligand is a ligand for the Angiotensin II receptor comprising the sequence Tyr-Pro-Phe.

In one embodiment, the ligand has the sequence Asp-Arg-Val-Tyr-Ile-Tyr-Pro-Phe.

In a further embodiment, lithe ligand can have a 4-substituted Phe residue in which an electron-donating or an electron-withdrawing group is introduced to substitute the hydrogen in the para-position of the phenylalanine ring.

For example, the ligand can have the sequence Asp-Arg-Val-Tyr-Ile-Phe-Pro-Phe wherein Phe⁶ is substituted at the 4 position.

In one embodiment, the ligand is provided for use as a negative regulator in the growth of pancreatic carcinoma cells through AT2R signaling²⁰. In a further embodiment, the ligand is provided for use as a negative regulator in the growth of lung adenocarcinoma cells through AT2R signaling²¹.

In a preferred embodiment, the ligand is the [Y]⁶-AII ligand.

In a further aspect, there is provided a method for treating a tumour in a patient in need of tumour therapy, comprising administering to said patient a pharmaceutically effective amount of a ligand as set forth in the foregoing aspects of the invention.

In a still further aspect, the ligand as set forth in the foregoing aspects of the invention is provided in combination with an Angiotensin I antagonist for the treatment of tumours. In one embodiment, a ligand according to the preceding aspects of the invention and an AT1 antagonist are provided for simultaneous, simultaneous separate or sequential use in the treatment of tumours,

Moreover, there is provided a kit comprising a ligand according to the preceding aspects of the invention and an AT1 antagonist, together with one or more pharmaceutically acceptable diluents or carriers.

An exemplary AT1 antagonist is Losartan.

### FIGURES

FIGS. 1a-b. [Y]⁶-AII shows enhanced cis isomerisation in solution. a) Schematic of cis-trans isomerization about the prolyl Tyr⁶-Pro⁷ bond. b) Selected region of a 350 ms NOESY spectrum of [Y]⁶-AII (90% H₂O/10% D₂O). The red and green lines denote the NOE connectivities for the trans and cis isomers respectively.

FIGS. 2a-d. Solution structures of the distinctive cis and trans conformers of the engineered AII analogue. a, b) Deconvolution of the two conformers was achieved on the basis of the high quality of chemical shift dispersion. c) NH Δδ/ΔT vs CSD for [Y]⁶-AII. The dashed line corresponds to Δδ/ΔT=−7.8 (CSD) −4.4, which provides the optimum differentiation of sequestered NHs in the protein database. d) ¹H DOSY spectrum of [Y]⁶-AII.

FIGS. 3a-b. Analogue selectivity: binding of analogues to AT1R, AT2R wild-type and mutants. Competition binding assays of [Y]⁶-AII (a) and [4-OPO3H2-F]⁶-AII (b) analogues to AT1R (black), AT2R wild type and mutants: AT1R, open circle; wild type AT2R, black circles; AT2R-Y189A, blue diamonds; AT2R-Y189N, green triangle; AT2R-F272A, red square; AT2R-F272H, orange triangle. Binding of [¹²⁵I]-ATII in the absence of unlabeled ligand was set to 100%. Data shown are from two independent experiments with each data point measured in triplicate. K_{D} and Kᵢ values are given in Table 4.

FIG. 4. Agonistic effect of the [Y]⁶-AII analogue for AT2 receptor: increased neurite outgrowth in AT2 over-expressing PC12W cells. PC12W cells, either transduced with the Ad-AT2R or untransduced, were seeded in a 24 well plate and cultured in 10% FBS containing DMEM for 24 hours. Culture medium was changed to 5 mg/ml bovine serum albumin (BSA) containing DMEM. Three hours later, cells were stimulated with either 1 nM AII or [Y]⁶-AII. Twenty-four hours later, 15 photos per well were taken. Five photos were randomly selected and cells showing neurite outgrowth were counted. Rate of the cells with neurite outgrowth to total cells were calculated. The neurite outgrowth cells were defined as the cells with the neurite length longer than its cell size. This experiment was carried out in triplicates. Data are expressed as mean±SE values.

FIG. 5. Mechanistic basis for the regulation of ligand selectivity for the AT2 and AT1a receptor subtypes. This was achieved by tuning of cis-trans isomerization and aromatic-prolyl interactions by aromatic electronics. The secondary structure of cis-trans isomerization about the prolyl 4-substituted Phe⁶-Pro⁷ bond is illustrated. The para substitution of Phe⁶ is indicated as X.

FIG. 6a-b. a) Mapping of the conserved residues between AT2R and AT1R in the homology model of AT2R. The figure was prepared with MOLMOL and ProtSkin. b) Mapping of the homologous residues of AT2R and other GPCRs in the homology model of AT2R. The figure was prepared with Rasmol and Protskin. Conserved residues between AT1R and AT2R exist in the TM regions. The majority of these residues overlay with homologues residues of other GPCRs.

FIG. 7. Indicative members of the families of clusters for the cis cases in the Tyrⁱ⁻¹-Proⁱ-Phe^{i|1} motif. From left to right are illustrated: the case of three ring clustering among Tyrⁱ⁻¹, Proⁱ and Pheⁱ⁺¹; two ring clustering between Tyrⁱ⁻¹ and Proⁱ; and two ring clustering between Tyrⁱ⁻¹ and Phe^{i 1} respectively. The most populated cluster is the three ring clustering among Tyrⁱ⁻¹, Proⁱ and Pheⁱ⁺¹ (table 3).

FIG. 8. In the X-ray structure of the complex between ubiquitin-protein ligase E3A and ubiquitin conjugating enzyme E2 (pdbid: 1C4Z), a Tyr-Pro-Phe motif (YPF), belonging to E2 (residues 61-63), is located in the interface of the interaction. In this motif (colored in red) there is a cis proline and its ring is packed against the aromatic rings of Tyr and Phe. The environment around the YPF motif was selected with a radius cut off of 6 Å (carbon colored in grey, nitrogen in blue and oxygen in red color). Interestingly, this environment closely resembles the environment near the ligand binding site of AT2R (residues colored in orange). The structure of AT2R used for this superposition was constructed based on the rhodopsin in its ligand-free state (pdbid: 3CAP). Homologues residues between AT2R and residues surrounding the environment of the Tyr-Pro-Phe motif are: W269/W105; K215/R96; Y189/Y694, Y51 (Al 94)/F698, L97/L695, L124/L696, L305(1304)/L659, T276/S65, I196/I697, L124/L696, P271/P58, L190/Y694, H273/F66, F220/P68. Phe308, Phe129, Phe272 and Ile304 could assist the assembly of a similar motif in AT2.

FIGS. 9a-b. Overlay of selected regions of 2D ¹H—¹H NOESY spectrums of [Y⁶]AII (colored black) and native AII (colored red) recorded under the same experimental conditions (0.01 M KPi buffer pH=5.7, 10% D₂O, 277K). The red and green lines denote the NOE connectivities for the trans and cis isomers respectively of [Y⁶]AII and the blue lines for the single trans isomer for the native AII.

FIG. 10. Region of the 750 MHz NOESY spectrum showing the intraresidue NOEs in the cis proline ring.

FIG. 11. Region of the 750 MHz NOESY spectrum showing the intraresidue C^{α}H—C^{β}H cross peaks for cis Tyr⁶ and cis Phe⁸, as well as a number of characteristic NOEs of the folded conformation of the cis form of the peptide.

### DETAILED DESCRIPTION OF THE INVENTION

Unless defined otherwise, all technical and scientific terms used herein have the same meaning as commonly understood by those of ordinary skill in the art, such as in the arts of peptide chemistry, cell culture, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.). All publications cited herein are incorporated herein by reference in their entirety for the purpose of describing and disclosing the methodologies, reagents, and tools reported in the publications that might be used in connection with the invention.

A selective ligand is a ligand which is capable of binding preferentially to a first receptor over a second. In the context of the present invention, a ligand is selective for one or another form of the angiotensin II receptor if it binds preferentially to that form; for example, the ligand may bind preferentially to AT1R over AT2R. Preferential binding does not imply exclusive binding, and the ratio of occupation of one form of the receptor over the other can vary anywhere between low (for example, 55% to 60% occupation of the desired receptor) to high (such as 95 to 100% occupation of the desired receptor. In most situations the ligand will be distributed between both forms of the receptor, and the ration of distribution will depend on a number of factors. These include not only the selectivity of the ligand, but also the concentration of the ligand relative to the receptor and the relative concentrations of receptor present.

The angiotensin II receptor is a well-characterised target for antihypertensive agents. Angiotensin receptor antagonists are widely used in cardiac medicine and the regulation of blood pressure. At least four types of the angiotensin II receptor are known, labelled AT1R through AT4R. The all bind the ligand angiotensin II. The present invention provides a means for creating ligands which are selective for one receptor subtype over another. This can have important physiological consequences; for example, as noted above, that many conditions are reported to be differentially influenced by AT1R and AT2R.

In the context of the present invention, cis-trans isomerisation is the formation of cis or trans isomers about the peptide bond between two amino acids in a polypeptide. Most peptide bonds adopt the trans isomer (typically 99.9% under unstrained conditions), largely because the amide hydrogen offers less steric repulsion to the preceding C^{α} atom than does the following C^{α} atom. In contrast, the cis and trans isomers of the X-Pro peptide bond (where X represents any amino acid) both experience steric clashes with the neighboring substitution and are nearly equal energetically. Hence, the fraction of X-Pro peptide bonds in the cis isomer under unstrained conditions ranges from 10-40%; the fraction depends on the preceding amino acid, with aromatic residues favoring the cis isomer. Pro can be replaced by N-substituted amino acids such as Sarcosine, but is unique amongst natural amino acids.

Protein databases which contain structural information, including cis-trans isomerism information, are widely accessible. One example is the protein databank (pdb) database, which contains structural information on a large variety of biomolecules.

A motif in an angiotensin II receptor ligand can be any sequence of amino acids which comprises the sequence X-Pro. Preferred ligands from which motifs can be derived are based on angiotensin. However, other polypeptide ligands for the angiotensin II acceptor can be envisaged, and motifs comprising the sequence X-Pro may be identified therein and used in the methods of the present invention.

Angiotensin is a peptide hormone derived by the cleavage of angiotensinogen, a 452 amino acid polypeptide which is cleaved by the action of renin to release the 10-amino acid polypeptide angiotensin I. This is further cleaved to form angiotensin II, the biologically active hormone, by cleaving off the two C-terminal residues. Further cleavage produces angiotensin III and IV by cleaving off one N-terminal residue in each case.

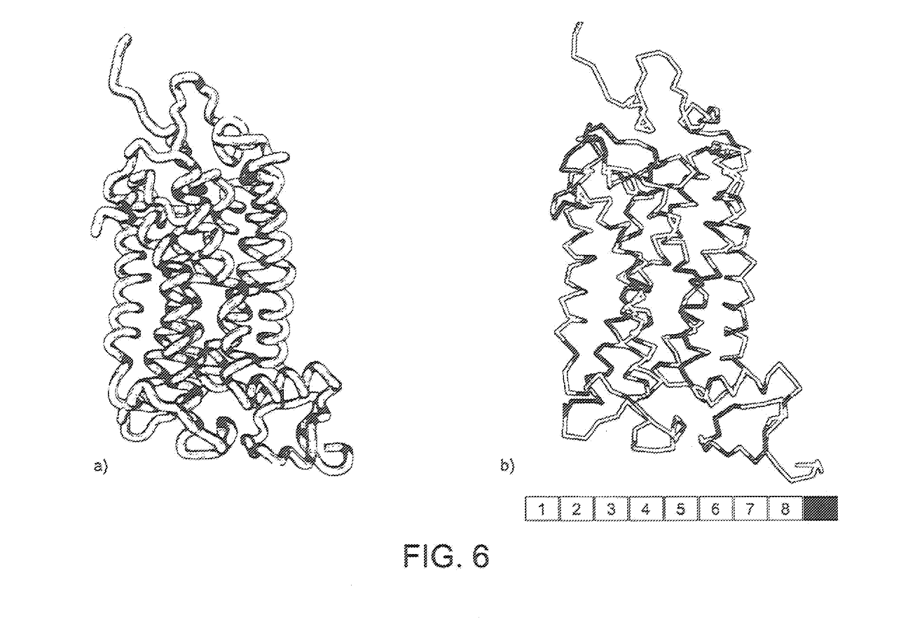

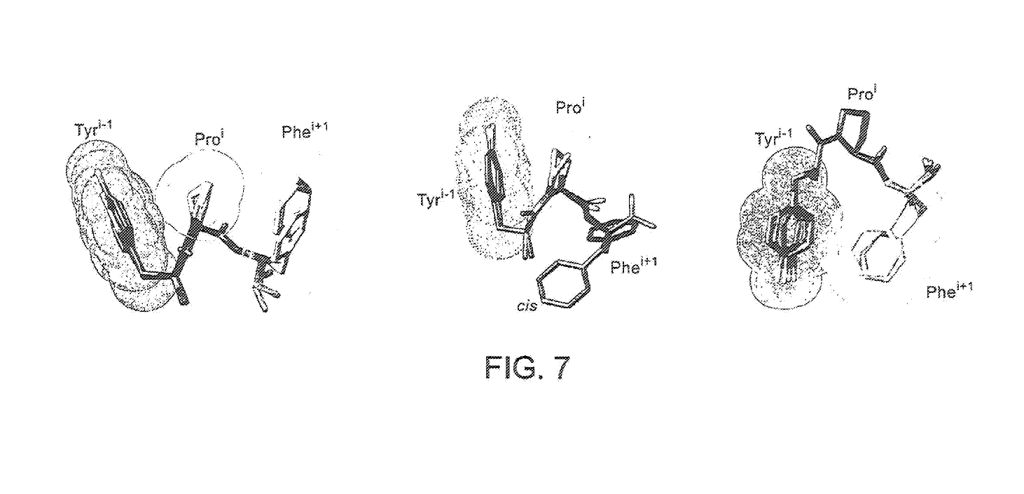

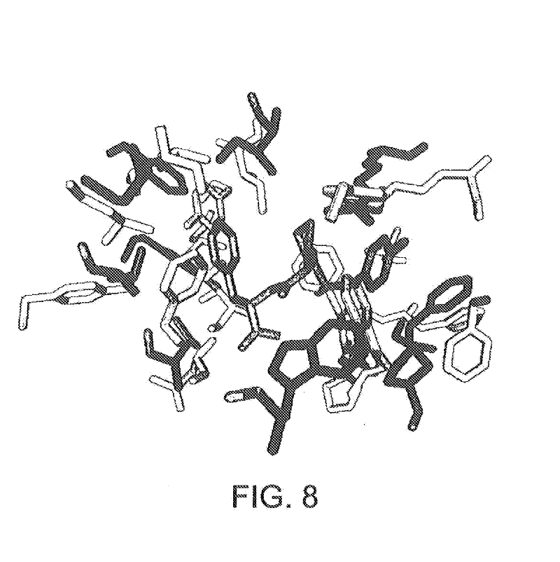

Engineering the hormone AII to be selective for AT2R. To investigate the role of proline in receptor selectivity, we focused on the C-terminus portion of the AII hormone (His⁶-Pro⁷-Phe⁸) which has been mapped through mutagenesis studies to dock deeply inside the AII receptors²³⁻²⁶. The protein structure database (www.pdb.org) was searched for X-Pro-Phe motifs (where X is any amino acid) to identify amino acid motif(s) with marked cis character (Table 1). It was evident that a great structural plasticity can be adopted in such motifs, depending on the amino acid X that precedes the proline (Data not shown). The AT1aR and AT2R ligand binding pockets were modeled based on residue conservation with known X-ray structures of other GPCRs and refined according to available mutagenesis studies (FIG. 6). According to this model, the residues that could be primarily responsible for determining ligand binding affinity and selectivity for AT2R/AT1aR, respectively, were the following: L124/V108, F308/Y292, L305/C289, F120/A104, T125/S109, F272/H256, G121/S105, F199/Y184, F129/Y113 and Y189/N174. The majority of these amino acids introduce more hydrophobic and larger residues near the mapped ligand binding site of AT2R relative to AT1aR, thus making it shallower. A similar observation was noted for the beta adrenergic receptor subtypes where ligand selectivity of b2AR relative to b1AR was assigned to be due to polar residue alteration¹. It could thus follow that a more hydrophobic and compact motif should be sought for an analogue to be selective for AT2R. From the X-Pro-Phe protein database in Table 1 the Tyr-Pro-Phe minicore seemed ideal since the cis state is highly populated and most of the structures containing this motif present a hydrophobic ring packing of the bulky aromatic side chains around the proline, leading to compactness (FIG. 7). We postulated that such residue packing could reduce the accessibility of the peptide bonds within the minicore, thus lowering the high cost of partitioning them into the more hydrophobic environment of the AT2R ligand-binding pocket. Analysis of the environment surrounding the Tyr-Pro-Phe minicore in the X-Pro-Phe database indicated a close resemblance to the environment present near the ligand binding pocket of the homology modeled AT2 receptor (FIG. 8). This suggests that the specific minicore could potentially be assembled and accommodated in the AT2R. Therefore, an AII analogue was synthesized by introducing a tyrosine residue instead of histidine at position 6 ([Y]⁶-AII: Asp¹-Arg²-Val³-Tyr⁴-Ile⁵-Tyr⁶-Pro⁷-Phe). Additionally, the substitution Tyr⁶ was preferred to Phe⁶ because the former has a higher electron-rich character that would better stabilize a C—H-π prolyl-aromatic interaction, thus favouring the more compact conformation of the cis state²⁷.

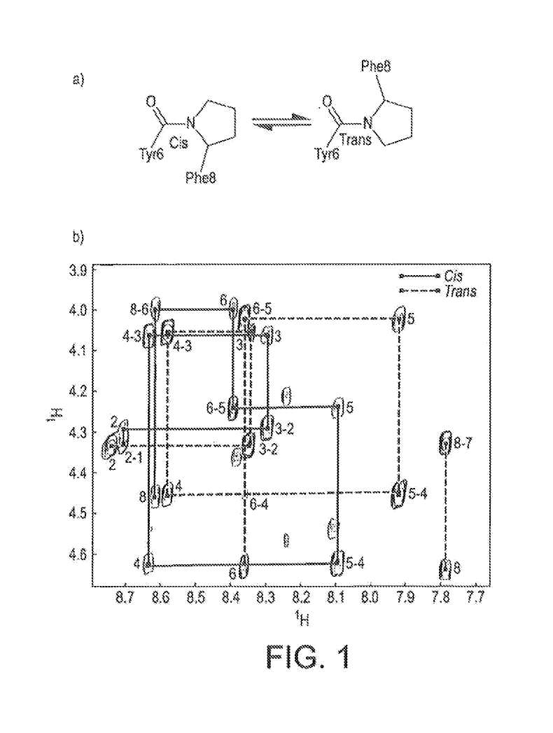

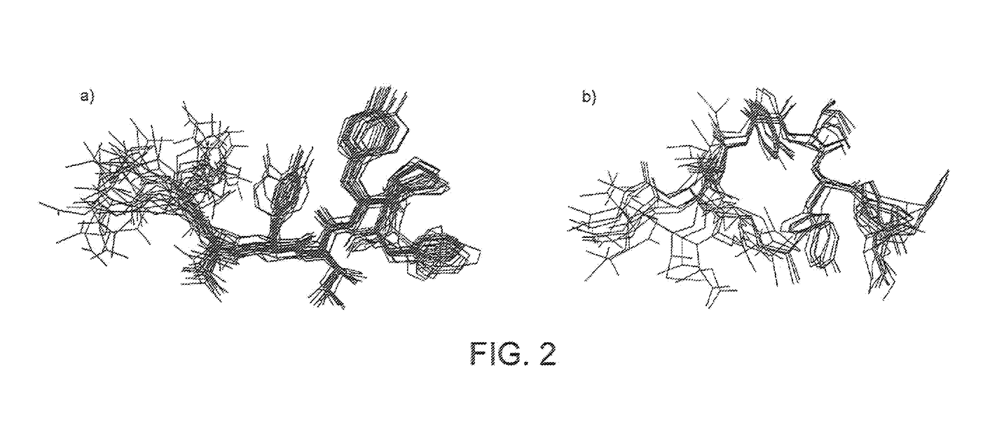

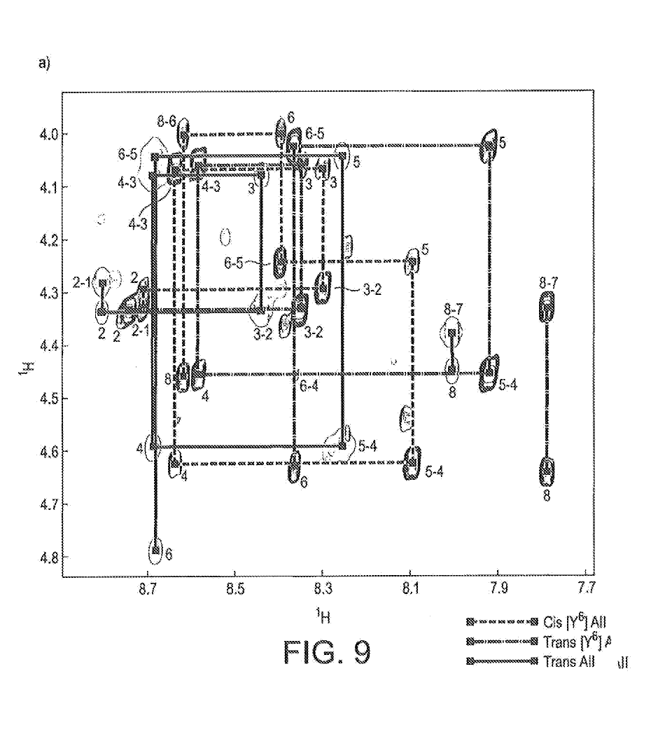

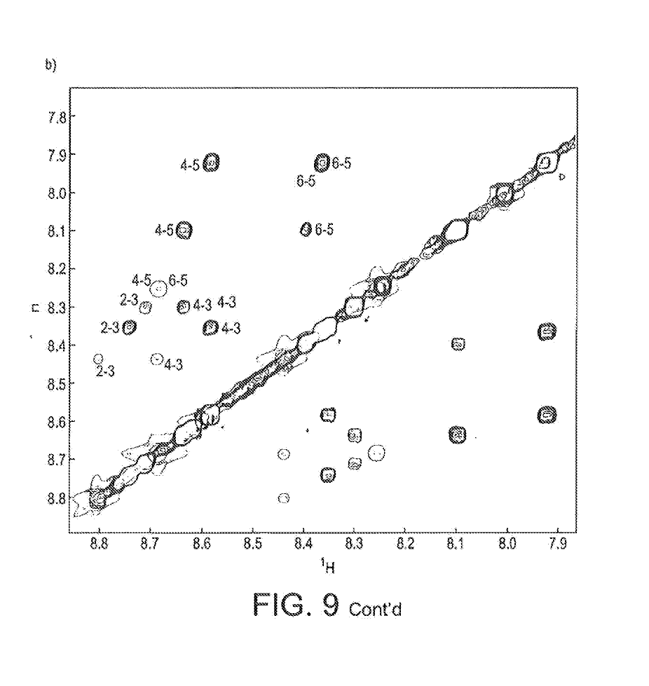

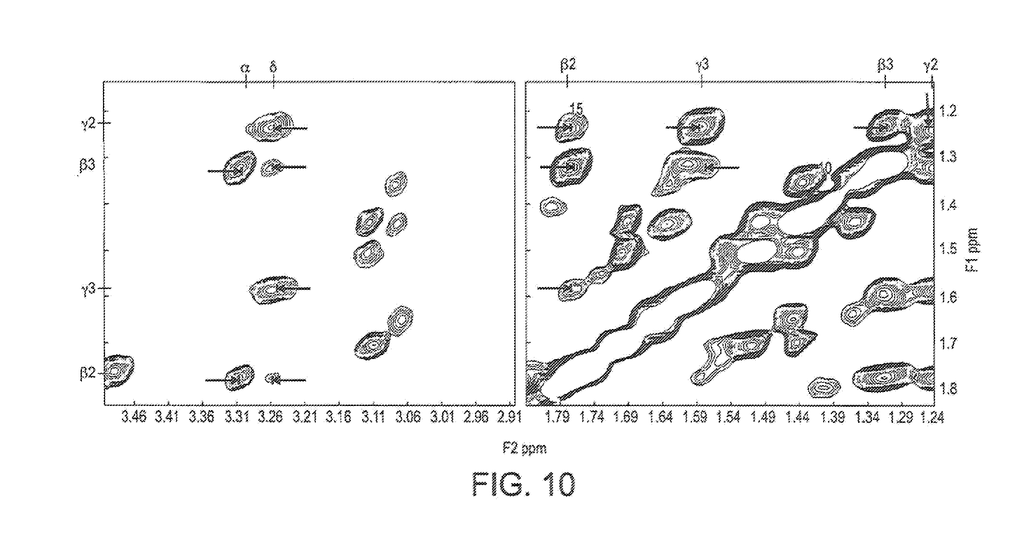

The [Y]⁶-AII shows enhanced cis isomerisation in solution. NMR was used to probe the [Y]⁶-AII analogue structure in solution. A selected region of the ¹H—¹H 2D NOESY spectrum of the analogue is shown in FIG. 1. Interestingly, [Y]⁶-AII shows two distinct sets of proton resonances that correspond to discrete cis and trans conformational populations in aqueous solution. This is in contrast to the native AII where a single set of peaks was observed, representing the single conformer (trans) (FIG. 9).

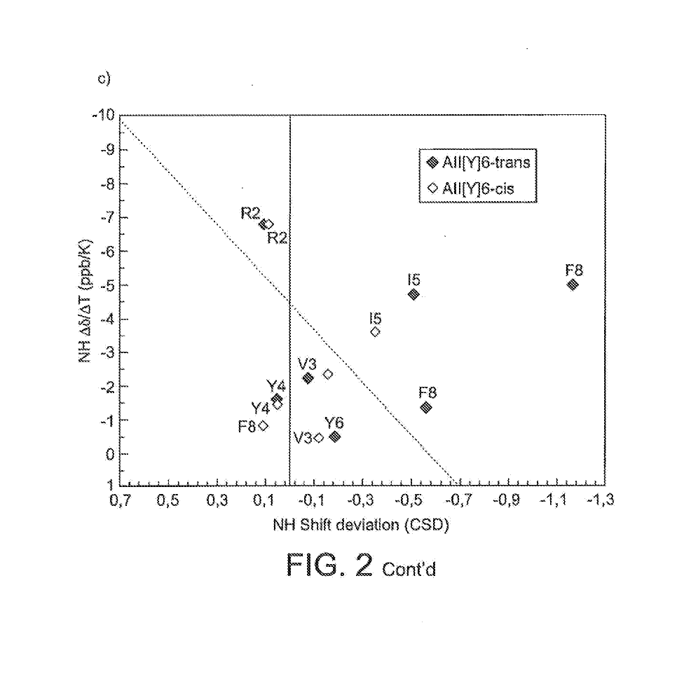

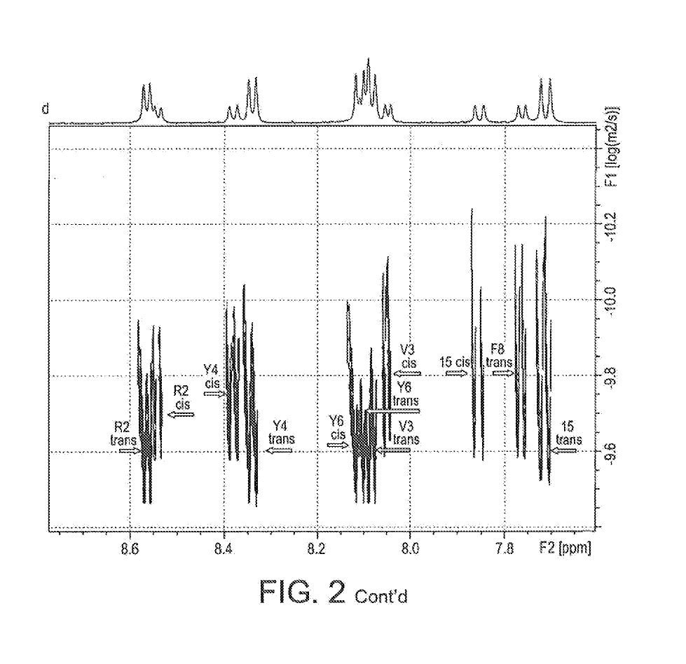

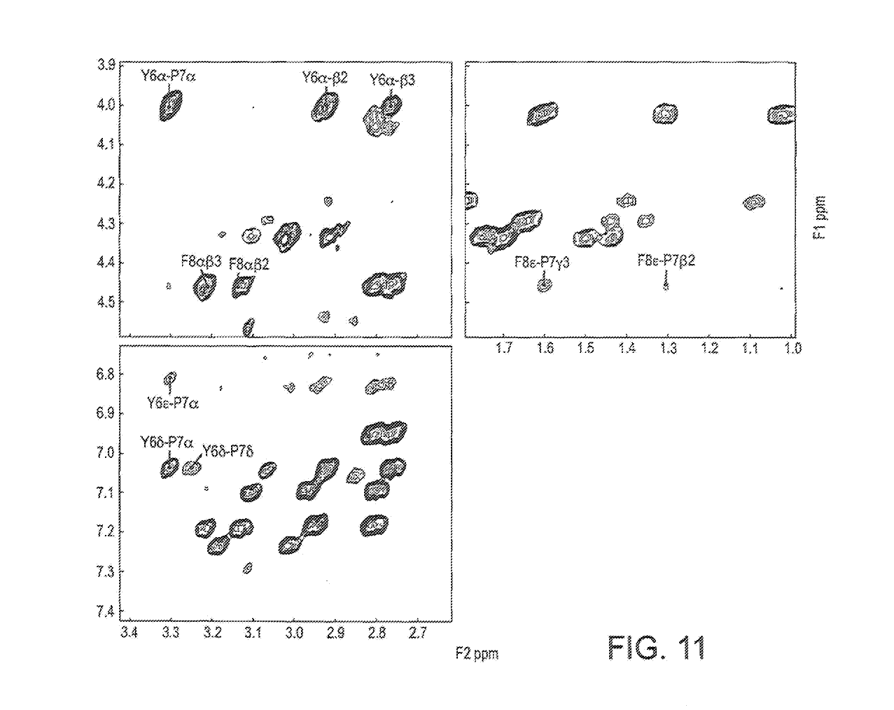

Due to excellent dispersion of the resonances of the cis and trans conformers, deconvolution and complete resonance assignment was achieved (Tables 2 and 3). NOE restraints were then selected to contain information only from the relevant members of the conformational ensemble. Structure calculations for the distinctive cis and trans isomers were performed and the structural origin of the stabilization of the relevant conformational potencies was mapped. For the [Y]⁶-AII cis isomer the calculations gave a family of structures with the segment Asp¹ to Ile⁵ being extended. The region Tyr⁶ to Phe⁸ showed a type VI turn with the aromatic rings of Tyr and Phe stacked against the Pro ring (FIG. 2a). The structural architecture of the Tyr-Pro-Phe minicore for the cis state mimics closely the conformation adopted by the major family recorded in the X-Pro-Phe protein database (FIG. 7). It is therefore evident that structural plasticity in short peptide sequences can be regulated by transferring information from protein motifs. The presence of the type VI conformation in the cis isomer is indicated by several features of the NMR spectrum. For instance, the significant upfield shifts of the proton resonances of the cis proline (Table 2 and 3); a cross-turn (i−i+2) NOE from residue 6 (Tyr⁶Hα) to residue 8 (Phe⁸NH); a C^{β}-exo/C^{γ}-endo conformation for the proline ring according to the pattern of intraresidue NOEs; an increased mole fraction of the cis form in the conformational ensemble (FIG. 11). The major stabilizing factor of this motif in the cis conformer is the stacking of the aromatic and proline rings. The structure of this motif in the [Y]⁶-AII trans conformer is more extended (FIG. 2b).

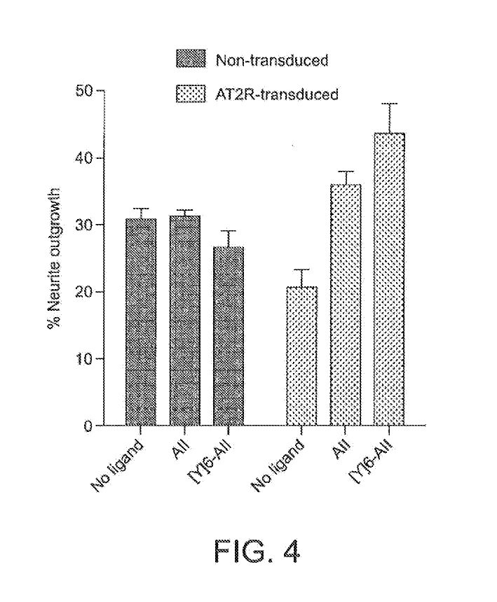

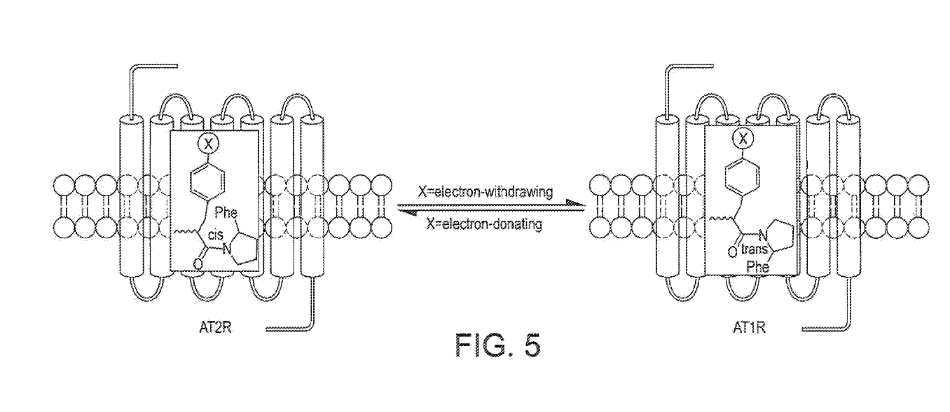

In order to determine the accessibility of the peptide bonds in the cis and trans forms, we measured both amide proton temperature coefficients (Δδ/ΔT) (FIG. 2c) as also their translational diffusion in solution by NMR. Diffusion coefficients were determined using a pulse-field gradient (PFG) technique, Diffusion Optimised Spectroscopy DOSY (diffusion-ordered spectroscopy)^{28, 29}. Interestingly, we found that the [Y]⁶-AII cis conformer has a more reduced accessibility of the peptide bonds compared to that of the trans conformer as determined both from the temperature coefficients and translational diffusion values (i.e. for Tyr⁴ we determined a diffusion coefficient of 1.9 10⁻¹⁰ m² s⁻¹ for the cis and 2.3 10⁻¹⁰ m² s⁻¹ for the trans, see also FIG. 4d). This reduction could allow the cis form to adopt a lower high cost of partitioning in the hydrophobic environment of AT2R ligand-binding pocket, in opposition to the trans form.

The [Y]⁶-AII analogue is selective for AT2R: importance of the cis character for receptor selectivity and affinity. Since the [Y]⁶-AII analogue fulfills experimentally the criteria to be selective for AT2R, we measured its binding to the AT1aR and AT2R. Interestingly, the analogue bound the AT2R with high affinity (Ki=3.4±0.8 nM), whereas we could not observe any saturable binding to the AT1aR in the submillimolar range of analogue concentration used. In order to elucidate whether the [Y]⁶-AII selectivity for AT2R was based on the increased cis character of the ligand and the resulting compactness of the Tyr-Pro-Phe motif, an electronic strategy²⁷ was adopted to control the cis-trans isomerization state of the AII analogues. Specifically, the aromatic-prolyl interaction can be stabilized not only due to the hydrophobic effect, but also by a C—H-π interaction, where the aromatic ring donates electron density (i-electron donor) to the electron deficient C—H bonds of the pyrrolidine ring²⁷. Therefore, electron-rich aromatic residues could stabilize the aromatic-prolyl interaction promoting the cis conformation. In contrast, electron-deficient aromatic residues should favour the trans conformation and lead to a less favourable interaction and compactness. We therefore, synthesized AII analogues introducing at position 6 4-substituted phenylalanine with electron-rich (—OH), electron-neutral (—H and —OPO₃H₂) and electron-deficient (—NO₂) groups. The 4-NO₂-phenylalanine AII analogue, that is an electron-deficient aromatic residue (value of Hammel substituent constant σp=0.78), should mostly disfavour the cis conformation whereas phenylalanine and phospho-tyrosine (σp≈0.00) should have moderate cis conformation. In contrast, tyrosine, an electron-rich aromatic residue (σ≈−0.37), favoured the ligand cis amide bond as experimentally determined by NMR. Indeed, NMR data indicated that electron rich residues favoured the aromatic-prolyl interaction and the cis amide bonds, with the following ranking order of aromatic substituents: —OH>—H≈—OPO₃H₂>—NO₂ (The %cis was found to be app. 40, 20, 25, and 5, respectively). On the basis of this control of cis-trans isomerism in AII via electronic tuning of the aromatic-prolyl interaction we then performed binding experiments of the analogues to the AT1aR and AT2R. Interestingly, the binding affinity and selectivity of all the AII analogues was directly correlated to the cis-trans isomerism of the analogues as described above (the rank order of affinities for the AT2R is: [Y]⁶-AII>[4-OPO₃H₂—F]⁶-AII>[F]⁶-AII>[4-NO₂—F]⁶-AII).

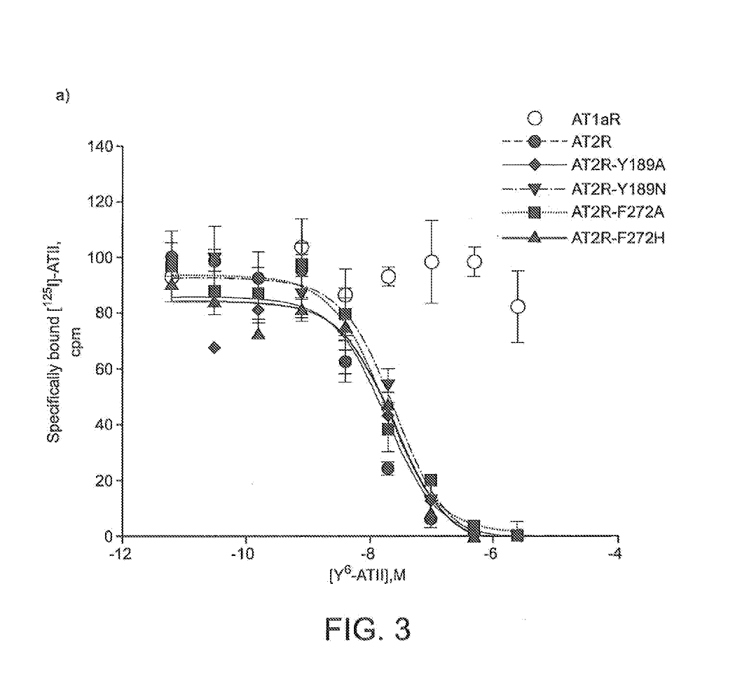

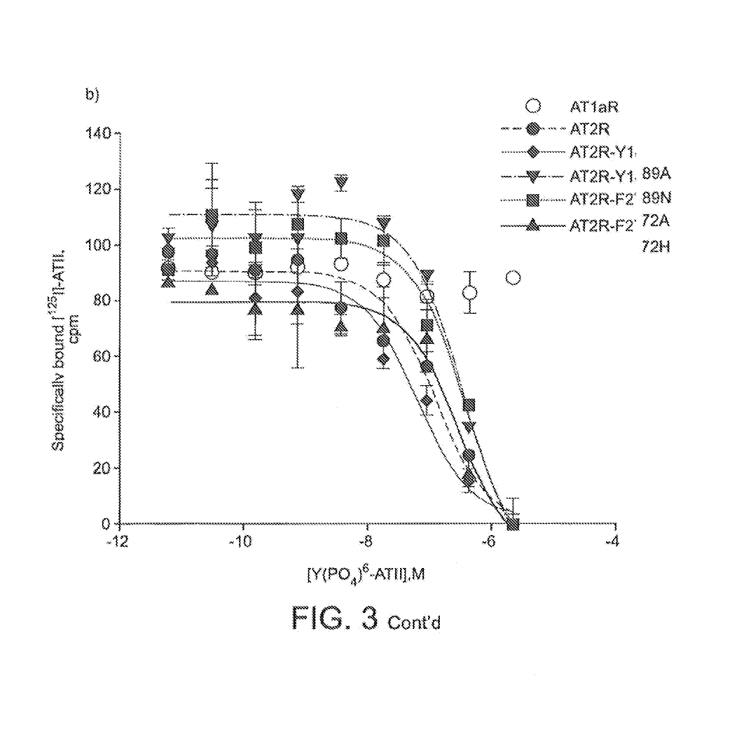

Potential location of the [Y]⁶ AII analogue in the AT2 receptor. Several mutagenesis studies indicated that the C-terminus part of AII is positioned deep inside the AT1aR^{23, 26, 30, 31} and AT2R^{26, 32, 33}. The Class A-GPCRs X-ray structures provided valuable templates to build realistic structural models of related receptors³⁴. As we mentioned above, reconstructed models of the AT2R and AT1aR suggested a shallower and more hydrophobic ligand binding site for AT2R compared to AT1aR for the C-terminal part of AII, indicating that a more compact C-terminus for AII could be required for high affinity binding to AT2R. In order to identify residues responsible for determining affinity and selectivity of the analogue [Y]⁶-AII for AT2R/AT1aR, we constructed the following AT2R mutants: Y189A, Y189N, F272A, and F272H. These changes introduce polar residues or residues of smaller size near the ligand binding pocket, emulating the more polar environment in the AT1aR ligand binding pocket. Both the analogues [Y]⁶-AII and [4-OPO₃H₂—F]⁶-AII were used to probe the binding pocket of AT2R and its mutants: the results are summarised in FIG. 3 and Table 4. The [Y]⁶-AII analogue appears to require an aromatic ring both in position 189 and 272 for optimal ring stacking in AT2R. As expected, the increased polarity of [4-OPO₃H₂—F]⁶-AII resulted in Ki values for AT2R one order of magnitude larger than the [Y]⁶-AII ligand. Compared to the wild type AT2R, the Y189N mutant displayed a decrease in affinity to [4-OPO3H2-F]⁶-AII probably due to the increased polarity and/or size of the side chain. For the same ligand, the substitution of the Tyr is position 189 with an Ala, a non-polar aminoacid, is better tolerated than the introduction of an asparagine. The mutant receptor F272A presents lower affinity than the F272H variant, suggesting a stabilizing ring stacking interaction between the imidazole group of the receptor variant and the tyrosine of the analogue through van der Waals contacts. Overall, these data support the initial hypothesis that the [Y]⁶-AII selectivity for AT2R is based on a more hydrophobic binding pocket (compared to AT1aR) that stabilizes the aromatic ring pairing between the Tyr⁶ of the analogue and the Y189/F272 residues in the AT2R binding pocket

The [Y]⁶-AII analogue is an AT2R agonist: it induces neurite outgrowth in PC12W cells over-expressing AT2R. To evaluate the effect of the [Y]⁶-AII analogue on cell differentiation (neurite outgrowth), PC12W cells were used. PC12W rat adrenal pheochromocytoma cells have a rounded shape and divide actively in the undifferentiated state. PC12W cells have been shown to be capable of expressing AT2R in lengthy serum-free culture condition³⁵ and their neurite outgrowth is stimulated by AII³⁶. PC12W cells did not express AT1aR in the current assay conditions as measured by the real time PCR (data not shown). As shown in FIG. 4, many cells have developed short neurites without stimulation. However, both AII and the [Y]⁶-AII analogue significantly stimulated neurite outgrowth in the AT2R transduced cells (FIG. 4). This phenotype was ligand dose-dependent in the range of 1 pM-100 nM for both AII and [Y]⁶-AII.

The [Y]⁶-AII analogue inhibits tumour cell proliferation but promotes would healing To assess the effect of AII analogues on tumour cell proliferation, three AII analogues were tested in a cell proliferation assay. The analogues used were A1 (sequence: DRVYICPF), with a cysteine residue at position 6; A2 (sequence: DRVYIdYPF), with a D-Tyr at position 6, and A3 (the [Y]⁶-AII analogue). The proliferation assay was performed in different cancer cells. See Table 5. A3 presented the best results in all studied cell lines, presenting excellent IC50 values in the nM range.

Data obtained with cell lines in which ATR1 expression has been silenced illustrate that reduction of ATR1 expression, or AT1(AI) antagonism, acts in complement with the AII analogues of the present invention in the inhibition of tumour cell proliferation. Data indicate that the AT1 antagonist Losartan and the AII analogues of the invention have highly beneficial combined effects.

Additional tests in a wound healing assay again showed that the [Y]⁶ analogue displays excellent IC50 values (Table 5).

TABLE 1
Structural statistics for the relative occurrence of cis form in
X-Pro-Phe peptide motifs in the PDB.

| X = aminoacid | Number_cis | Number_trans | % cis | % trans |
|---|---|---|---|---|
| PRO | 53 | 203 | 20.70% | 79.30% |
| GLY | 62 | 291 | 17.56% | 82.44% |
| TYR | 35 | 223 | 13.57% | 86.43% |
| TRP | 8 | 57 | 12.31% | 87.69% |
| PHE | 22 | 184 | 10.68% | 89.32% |
| GLU | 37 | 318 | 10.42% | 89.58% |
| ALA | 38 | 399 | 8.70% | 91.30% |
| LYS | 38 | 402 | 8.64% | 91.36% |
| SER | 27 | 344 | 7.28% | 92.72% |
| GLN | 13 | 227 | 5.42% | 94.58% |
| HIS | 11 | 215 | 4.87% | 95.13% |
| ARG | 15 | 309 | 4.63% | 95.37% |
| THR | 18 | 377 | 4.56% | 95.44% |
| CYS | 3 | 79 | 3.66% | 96.34% |
| LEU | 21 | 578 | 3.51% | 96.49% |
| VAL | 16 | 460 | 3.36% | 96.64% |
| ILE | 13 | 384 | 3.27% | 96.73% |
| ASP | 9 | 287 | 3.04% | 96.96% |
| MET | 4 | 128 | 3.03% | 96.97% |
| ASN | 9 | 314 | 2.79% | 97.21% |

TABLE 2
Complete resonance assignment for the trans isomer of the AII analogue.

| | | |
|---|---|---|
| 1.HA | 4.332 | 1 |
| 1.HB2 | 3.030 | 2 |
| 1.HB1 | 2.916 | 2 |
| 2.HN | 8.741 | 1 |
| 2.HA | 4.333 | 1 |
| 2.HB2 | 1.699 | 2 |
| 2.HB1 | 1.699 | 2 |
| 2.HG2 | 1.501 | 2 |
| 2.HG1 | 1.435 | 2 |
| 2.HD2 | 3.106 | 2 |
| 2.HD1 | 3.106 | 2 |
| 2.HE | 7.101 | 1 |
| 2.HH21 | 6.877 | 1 |
| 2.HH22 | 6.877 | 1 |
| 2.HH11 | 6.429 | 1 |
| 2.HH12 | 6.429 | 1 |
| 3.HN | 8.354 | 1 |
| 3.HA | 4.050 | 1 |
| 3.HB | 1.933 | 1 |
| 3.HG21 | 0.886 | 2 |
| 3.HG11 | 0.832 | 2 |
| 4.HN | 8.582 | 1 |
| 4.HA | 4.455 | 1 |
| 4.HB2 | 2.765 | 2 |
| 4.HB1 | 2.807 | 2 |
| 4.HD1 | 6.948 | 3 |
| 4.HE1 | 6.641 | 3 |
| 5.HN | 7.921 | 1 |
| 5.HA | 4.021 | 1 |
| 5.HB | 1.605 | 1 |
| 5.HG12 | 1.309 | 1 |
| 5.HD11 | 0.730 | 1 |
| 5.HG21 | 1.022 | 2 |
| 6.HN | 8.362 | 1 |
| 6.HA | 4.632 | 1 |
| 6.HB2 | 2.960 | 2 |
| 6.HB1 | 2.806 | 2 |
| 6.HD1 | 7.179 | 3 |
| 6.HE1 | 6.817 | 3 |
| 7.HA | 4.324 | 1 |
| 7.HB2 | 2.094 | 2 |
| 7.HG2 | 1.897 | 2 |
| 7.HG1 | 1.754 | 2 |
| 7.HD2 | 3.484 | 2 |
| 7.HD1 | 3.775 | 2 |
| 8.HN | 7.791 | 1 |
| 8.HA | 4.641 | 1 |
| 8.HB2 | 3.011 | 2 |
| 8.HB1 | 3.188 | 2 |
| 8.HD1 | 7.233 | 3 |
| 8.HE1 | 7.330 | 3 |

TABLE 3
Complete resonance assignment for the cis isomer of the AII analogue.

| | | |
|---|---|---|
| 1.HA | 4.332 | 1 |
| 1.HB2 | 3.030 | 2 |
| 1.HB1 | 2.916 | 2 |
| 2.HN | 8.706 | 1 |
| 2.HA | 4.293 | 1 |
| 2.HB2 | 1.648 | 2 |
| 2.HG2 | 1.437 | 2 |
| 2.HG1 | 1.349 | 2 |
| 2.HD2 | 3.074 | 2 |
| 2.HE | 7.042 | 1 |
| 3.HN | 8.297 | 1 |
| 3.HA | 4.067 | 1 |
| 3.HB | 1.914 | 1 |
| 3.HG21 | 0.865 | 2 |
| 4.HN | 8.635 | 1 |
| 4.HA | 4.625 | 1 |
| 4.HB2 | 2.798 | 2 |
| 4.HB1 | 2.965 | 2 |
| 4.HD1 | 7.092 | 3 |
| 4.HE1 | 6.752 | 3 |
| 5.HN | 8.093 | 1 |
| 5.HA | 4.239 | 1 |
| 5.HB | 1.793 | 1 |
| 5.HD11 | 0.850 | 1 |
| 5.HG21 | 0.910 | 2 |
| 6.HN | 8.394 | 1 |
| 6.HA | 3.997 | 1 |
| 6.HB2 | 2.763 | 2 |
| 6.HB1 | 2.922 | 2 |
| 6.HD1 | 7.035 | 3 |
| 6.HE1 | 6.815 | 3 |
| 7.HA | 3.304 | 1 |
| 7.HD2 | 1.771 | 2 |
| 8.HN | 8.619 | 1 |
| 8.HA | 4.459 | 1 |
| 8.HB2 | 3.129 | 2 |
| 8.HB1 | 3.219 | 2 |
| 8.HD1 | 7.198 | 3 |
| 8.HE1 | 7.321 | 3 |

TABLE 4
Summary of results for 125-I AII saturation assays and for analogues
competition assays of wild-type and mutant AII receptors.
Assays were carried out as described in Material and Methods. Incubation
of samples with ligands was for 2 hours on ice. The concentration of
[¹²⁵I]-AII used in competition binding assays was 2 nM.
K_{D} data were fitted to the Michaelis-Menten equation using the non-
linear regression equation of the software Prism. Ki values were calculated
according to the Cheng and Prusoff equation with a the KD values in the
first column. Values are representative of 2-3 independent experiments.
Each data point was assayed in triplicate.

| K_{D} (nM) | Ki (M) | |
|---|---|---|
| [¹²⁵I]-AII | [Y]⁶-AII | [4-OPO₃H₂—Y]⁶-AII |

| | | | |
|---|---|---|---|
| AT1R | 1.8 ± 0.1 | —^{a} | —^{a} |
| AT2R | 2.3 ± 0.3 | 3.4 × 10⁻⁹ | 9.3 × 10⁻⁸ |
| AT2R-Y189A | 7.0 ± 0.9 | 1.2 × 10⁻⁸ | 5.1 × 10⁻⁸ |
| AT2R-Y189N | 3.7 ± 0.5 | 1 × 10⁻⁸ | 2 × 10⁻⁷ |
| AT2R-F272A | 4.2 ± 0.3 | 1.6 × 10⁻⁸ | 2.5 × 10⁻⁷ |
| AT2R-F272H | 3.1 ± 0.6 | 1.6 × 10⁻⁸ | 2.1 × 10⁻⁸ |

^{a}No detectable competitive binding was measured in the ligand concentration range used (6.4 × 10⁻¹²-2.5 × 10⁻⁶M).

TABLE 5
Summary of IC50 data in tumour cell proliferation and wound healing
assays.

| | | | | |
|---|---|---|---|---|
| Cell line | AGTR1 status | IC50 A1 | IC50A2 | IC50 A3 |

Proliferation Assay

| | | | | |
|---|---|---|---|---|
| SKBR3 | Expressed | 10⁻⁵M | 10⁻⁵M | 10⁻⁶M |
| MB 231 | Silenced | 10⁻⁵M | 10⁻⁵M | 10⁻¹⁰M |
| MB 435 | Silenced | 10⁻⁵M | 10⁻⁵M | 10⁻⁹M |
| MB 436 | Expressed | 10⁻⁵M | 10⁻⁵M | 10⁻⁷M |
| MB 453 | Silenced | 10⁻⁵M | 10⁻⁵M | 10⁻⁹M |
| MB 468 | Silenced | 10⁻⁵M | 10⁻⁵M | 10⁻⁹M |
| MCF7 | Expressed | 10⁻⁵M | 10⁻⁵M | 10⁻⁶M |
| HCC1937 | Expressed | 10⁻⁵M | 10⁻⁵M | 10⁻⁷M |
| BT549 | Expressed | 10⁻⁵M | 10⁻⁵M | 10⁻⁷M |
| T47D | Expressed | 10⁻⁵M | 10⁻⁵M | 10⁻⁶M |
| ZR751 | Expressed | 10⁻⁵M | 10⁻⁵M | 10⁻⁶M |
| HS578 | Expressed | 10⁻⁵M | 10⁻⁵M | 10⁻⁷M |

Wound healing

| | | | |
|---|---|---|---|
| MB 231 | 10⁻⁵M | 10⁻⁵M | 10⁻⁹M |
| MCF7 | 10⁻⁵M | 10⁻⁷M | 10⁻⁸M |

### Pharmaceutical Compositions

In a preferred embodiment, there is provided a pharmaceutical composition comprising a compound or compounds identifiable by an assay method as defined in the previous aspect of the invention, including ligands as described above.

A pharmaceutical composition according to the invention is a composition of matter comprising a compound or compounds capable of specifically activating the AT2R as an active ingredient. Typically, the compound is in the form of any pharmaceutically acceptable salt, or e. g., where appropriate, an analog, free base form, tautomer, enantiomer racemate, or combination thereof. The active ingredients of a pharmaceutical composition comprising the active ingredient according to the invention are contemplated to exhibit excellent therapeutic activity, for example, in the treatment of tumours such as pancreatic cancer and lung cancer, when administered in amount which depends on the particular case. Exemplary compounds are AII analogues which comprise the sequence Tyr-Pro-Phe.

In another embodiment, one or more compounds of the invention may be used in combination with any art recognized compound known to be suitable for treating any of the aforementioned conditions. Accordingly, one or more compounds of the invention may be combined with one or more art recognized compounds known to be suitable for treating the foregoing indications such that a convenient, single composition can be administered to the subject. Dosage regimens may be adjusted to provide the optimum therapeutic response.

For example, several divided doses may be administered daily or the dose may be proportionally reduced as indicated by the exigencies of the therapeutic situation.

The active ingredient may be administered in a convenient manner such as by the oral, intravenous (where water soluble), intramuscular, subcutaneous, intranasal, intradermal or suppository routes or implanting (e. g. using slow release molecules).

Depending on the route of administration, the active ingredient may be required to be coated in a material to protect said ingredients from the action of enzymes, acids and other natural conditions which may inactivate said ingredient.

In order to administer the active ingredient by other than parenteral administration, it will be coated by, or administered with, a material to prevent its inactivation. For example, the active ingredient may be administered in an adjuvant, co administered with enzyme inhibitors or in liposomes. Adjuvants contemplated herein include resorcinols, non-ionic surfactants such as polyoxyethylene oleyl ether and hexadecyl polyethylene ether.

Liposomes include water-in-oil-in-water CGF emulsions as well as conventional liposomes.

The active ingredient may also be administered parenterally or intraperitoneally.

Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active ingredient in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredient is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active ingredient may be incorporated with excipients and used in the form of ingestible tablets, buccal tablets, troches, capsules, elixirs, suspensions, syrups, wafers, and the like. The amount of active ingredient in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the active ingredient of the invention as hereinbefore defined for use in the treatment of disease either alone or in combination with art recognized compounds known to be suitable for treating the particular indication. Consequently there is provided the use of an active ingredient of the invention for the manufacture of a medicament for the treatment of cancer, especially pancreatic or lung cancer, and methods of therapy associated with the same.

### Materials and Methods

Materials. AII and AT2R receptor-specific blocker PD123319 were purchased from Sigma-Aldrich Chemical Co. (St. Louis, Mo.). Human AGTR2 pcDNA3.1+ was obtained from the UMR cDNA Resource Centor (University of Missouri-Rolla, Rolla, Mo.). All other chemicals were of analytic grade. The AT1aR and AT2R constructs were a kind gift from Lazlo Hunyady (Semmelweis University, Budapest, Hungary;⁴³).

Mutagenesis. AT2R mutants were generated as described elsewhere⁴⁴.

Cell culture and transient transfection. HEK293T cells were maintained at 37° C. in Dulbecco's modified Eagle's medium (DMEM) supplemented with 10% (v/v) fetal bovine serum (FBS), 2 mM glutamine, 100 U/ml penicillin, and 100 μg/ml streptomycin. Cells were seeded into 10 cm dish and 24 hours later were transfected with either AT1aR (pcDNA3.1) or AT2R (pcDNAI/Amp) using GeneJuice transfection reagent according to the manufacturer's instructions. Cells were harvested 48 hours after transfection. A pellet of transfected HEK293T cells (2×10⁶ cells) was resuspended in ice-cold 0.5 ml binding buffer (50 mM Tris-HCl pH 7.4, 100 mM NaCl, 10 mM MgCl₂, 1 mM EDTA, 0.2% BSA and 0.025% Bacitracin) containing protease inhibitors (Complete™, Roche). The cells were transferred to a 1.5 ml microcentrifuge tube and subjected to two cycles of freeze-thawing. The lysed cells were sheared by passaging seven times through a 26-gauge needle and the crude membranes were pelleted by ultracentrifugation (60 min, 120,000×g, 4° C.). The crude membranes were then resuspended in a final volume of 0.2 ml ice-cold binding buffer containing protease inhibitors corresponding to a protein concentration 3.3 μg/μl as determined by the amido black protein assay⁴⁵.

PC12W cells, a substrain from a clonal isolation of a rat adrenal chromaffin cell tumor, were cultured in DMEM supplemented with 10% FBS, 100 units/ml penicillin and 100 μg/ml streptomycin (Invitrogen, Carlsbad, Calif.) as previously described⁴⁶. The cells were incubated in a 5% CO₂ humidified incubator at 37° C. Preparation of recombinant replication-deficient adenovirus containing the human AT2R coding region was carried out by VECTOR BIOLABS (Philadelphia, Pa.).

For gene transduction with adenoviral vectors cells were seeded at 1.2×10⁵ cells per well in a 6-well plate. After 24 hours, cells were incubated at 37° C. for 6 hours in serum-free DMEM containing adenoviral vectors (Ad-AT2R 30 multiplicity of infection (MOI)), and shaken lightly every fifteen minutes. After 6 hr incubation, cells were cultured in 10% FBS containing DMEM at 37° C., 5% CO₂ for an additional 24 hours. Cells were then trypsinized and subcultured at a density of 2×10³ cells per well on a 24-well plate. For the evaluation of the effect of [Y]⁶-AII analogue on the neurite outgrowth, at 24 hours after subculture, control cells and the AT2R over-expressing cells were treated with 1 nM AII or [Y]⁶-AII as indicated in FIG. 4.

Radioligand binding assays. Saturation curves were obtained using a range of [¹²⁵I]-AII (Amersham) concentration 0-10 nM (8 data points in triplicate). Non-specific binding was determined in presence of 6 μM cold AII. Competition assays were performed using a concentration of [¹²⁵I]-AII of 1 nM and various concentrations of unlabeled ligands, as indicated in the FIG. 3. Samples were incubated for 2 hours at 4° C. Receptor-bound and free radioligand were separated by filtration through Whatman GF/B filters, pre-soaked with 0.3% polyethylamine. The filters were washed with 5 ml of ice-cold binding buffer and transferred to scintillation tubes. Radioactivity was counted on a Beckman LS6000 liquid scintillation counter and data were analyzed by non-linear regression using Prism software (GraphPad). Kᵢ values were calculated according to the Cheng and Prusoff equation with a K_{D} for [¹²⁵I]-AII of 1.8 nM (AT1aR) and 2.3 nM (AT2R).

Peptide synthesis and sample preparation. The synthesis of the following peptides: AII (D¹-R²—V³—Y⁴—I⁵—H⁶—P⁷—F⁸); [Y]⁶-AII (D¹-R²—V³—Y⁴—I⁵—Y⁶—P⁷—F⁸); [4-OPO₃H₂—F]⁶-AII, (D¹-R²—V³—Y⁴—I⁵-(4-OPO₃H₂—F)⁶—P⁷—F⁸), [F]⁶-AII, (D¹-R²—V³—Y⁴—I⁵—F⁶—P⁷—F⁸) and [4-NO₂—F]⁶-AII, (D¹-R²—V³—Y⁴—I⁵-(4-NO₂—F)⁶—P⁷—F⁸) was achieved using Fmoc/tBu methodology. 2-Chlorotrityl chloride resin and N^{α}-Fmoc (9-fluorenylmethyloxycarbonyl) amino acids were used for the synthesis. Peptide purity was assessed by analytical HPLC (Nucleosil-120 C18, reversed phase, 250×4.0 mm), mass spectrometry (FABMS, ESIMS) and amino acid analysis. The samples were prepared for NMR spectroscopy by dissolving the peptide in 0.01 M KPi buffer (pH=4), containing 0.02 M KCl. 2,2-dimethyl-2-sila-pentane sulfonate (DSS) was added to a concentration of 1 mM as an internal chemical shift reference. Peptide concentration was commonly 4 mM in 90% ¹H₂O/10% ²H₂O. Trace amounts of NaN₃ were added as a preservative.

### NMR Spectroscopy

a) Determination of distance restrains. High field NMR spectra were acquired at 500 MHz using a Bruker Avance 500 spectrometer in the NMR center of the University of Ioannina and 750 MHz using a Bruker Avance 750 spectrometer in the Bijvoet Center for Biomolecular Research in Utrecht. For water suppression excitation sculpting with gradients was used. All proton 2D spectra were acquired using the States-TPPI method for quadrature detection, with 2K×512 complex data points and 16 scans per increment for 2D TOCSY and 64 scans for 2D NOESY experiments, respectively. The mixing time for TOCSY spectra was 80 ms. Mixing times for NOESY experiments were set to 100, 200, 350 and 400 ms to determine NOE build-up rates. A mixing time of 350 ms provided sufficient cross-peak intensity without introducing spin-diffusion effects in the 2D-NOESY. Phase-sensitive 2D NOESY was used for specific assignment and for estimation of proton-proton distance constrains. Data were zero filled in t₁ to give 2 K×2 K real data points, and 90° phase shifted square cosine-bell window function was applied in both dimensions. All spectra were processed by using NMRPipe software package and analysed with NMRVIEW.

Inter-proton distances for AII were derived by measuring cross-peak intensities in the NOESY spectra. Intensities were calibrated to give a set of distance constrains using the NMRVIEW software package. NOEs cross peaks were separated into three distance categories according to their intensity. Strong NOEs were given an upper distance restraint of 3.0 Å, medium NOEs a value of 4.0 Å and weak NOEs 5.5 Å. The lower distance limits were set to 1.8 Å. The mole fraction of the peptide molecules in the cis isomeric form (Xcis) was obtained by measuring the areas of well-resolved peaks corresponding to the same proton resonance in the cis and trans forms in 1D spectra.

b) Temperature coefficients. To investigate solvent protection values for amide protons, the amide proton temperature coefficients (Δδ/ΔT) were measured in a range of temperatures from 283K to 308K. Exposed NHs typically have gradients in the range of −6.0 to −8.5 p.p.b./K, whereas hydrogen-bonded or protected NHs apparently have Δδ/ΔT of −2.0 to ±1.4 p.p.b.·K^{-1 47}. A plot of Δδ/ΔT vs. the chemical shift deviation (CSD) of the measured amide proton resonances at 283 K (Figure X), with appropriate random coil chemical shift correction⁴⁸, provides a better correlation with partial structuring of a flexible linear peptide. The dashed line (Δδ/ΔT=−7.8 (CSD)−4.4) represents the cut off of Δδ/ΔT between exposed and sequestered NHs of proteins. Gradients above the dashed line indicate exposed NHs, whereas those below indicate sequestered NHs. As can be seen in FIG. 2, all the backbone NH, with the exception of the Arg2, are above the dashed line, indicating that these peptide protons are somewhat exposed. The Arg2 backbone NH is most probably implicated in the formation of an intramolecular hydrogen bond (see discussion below). Low Δδ/ΔT values for the backbone NH of Arg2 have been found in cyclic analogues of AII, suggesting shielding from the solvent, but with no rationalization about the structural origin of this effect.

c) Diffusion Ordered NMR spectroscopy. The Bruker microprogram stebpgp1s19 was applied to obtain diffusion ordered spectroscopy (DOSY) spectra at 298 K. The pulse-program applies stimulated echoes using bipolar gradient pulses for diffusion and 3-9-19 pulses with gradients for water suppression. For each FID, 512 transients were collected with 3 s relaxation delay and a 20 μs delay for binomial water suppression. 4096 data points in the F2 dimension (20 ppm) and 16-32 data points (gradient strengths) in the F1 dimension were collected for all experiments. Final data sizes were 4096×1024. Exponential multiplication was applied in F2 with 1 Hz line broadening. The diffusion time (Δ) and the gradient length (δ) were set to 100 ms and 1 ms, respectively, while the recovery delay after gradient pulses was 200 μs. Two types of data analyses were applied to the raw experimental data. For automatic 2D-processing, the standard 2D DOSY processing protocol was applied in XWINNMR software with logarithmic scaling in the F1 (diffusion coefficient) dimension. For manual curve-fitting, the intensities of selected peaks in the 1D proton spectra measured at different gradient strengths were fitted using the equation I=I₀exp(−Dγ²g²δ²(Δ−δ/3))[→sqrt(−ln(I/Io))=sqrt(D*)g] (A R Waldeck, P W Kuchel, A J Lennon, and B E Chapman, NMR Diffusion Measurements to Characterise Membrane Transport and Solute Binding. Prog. NMR Spectrosc.) to obtain the apparent diffusion coefficient D*. In this theoretical equation the following physical quantities are symbolized: I, the actual (measured) peak intensity; I₀, peak intensity at zero gradient strength; D, diffusion coefficient; γ, gyromagnetic ratio (of proton); g, gradient strength; δ, length of gradient; and Δ, diffusion time. Theoretically the length of gradient and the diffusion time can also be incremented in diffusion experiments, however, most pulse-schemes modify the gradient strength (g). Since D, γ, Δ and δ are constant, in Dγ²g²δ²(Δ−δ/3) they can be converted to be under a new constant, D* (D*=cD, where c=γ²δ²(Δ−δ/3) and is a constant). By mathematical rearrangement of the original equation and substitution of the new constant (D*), a linear equation is deduced [sqrt(−ln(I/Io))=sqrt(D*)g] (see above), that is applicable in determining the diffusion coefficient. On these plots, gradient strengths are represented as the linearly changing increments of the total gradient strength between 5% and 95% (16 or 32 increments were applied). As shown in the equation, the slope of the fitted line is equal to the square root of D*, so D* can be calculated from the value of the slope. The actual molecular weights relative to the references can thus be determined by the following equation, log(D₁/D₂)=⅓*log(MW₂/MW₁), where D1/D2=D1*/D2* (A R Waldeck, P W Kuchel, A J Lennon, and B E Chapman, NMR Diffusion Measurements to Characterise Membrane Transport and Solute Binding. Prog. NMR Spectrosc.) This equation assumes that the molecules being compared have the same overall shapes and relaxation properties.

Structure calculations. Structure calculations were performed with CNS using the ARIA setup and protocols, as described in Bonvin et al.

Construction and analysis of a structure protein dataset having the X-Pro-Phe sequence motif (X=any aminoacid). A dataset of protein structures from the Protein Data Bank [ref] (PDB) with <90% sequence identity threshold, resolution of ≦3.0 Å, and R-factors of ≦0.3 obtained from the PISCES server⁴⁹. Prolyl residues of 12736 protein structures were examined (see materials and methods) For each prolyl residue, the torsion angle omega (ω) was calculated. Bonds with an angle between −50° and +50° were considered as cis prolyl bonds. The database was further processed to avoid redundancy (i.e. residues present in different chains in the same pdb, but having same sequence were only kept if the difference in their torsion angle was greater than 50°), and missing neighboring residues for the prolines of interest. Home made scripts written in C⁺⁺ (Tsoulos I., Tzakos A. G., et al. in preparation) were used to: (i) calculate the torsion angle ω for the proline residues; (ii) construct a dataset of residues having the X-Pro-Phe motif (where X any aminoacid); (iii) calculate the statistics for the occurrence of the cis proline amide bond for every aminoacid in the X-Pro-Phe motif (Table 1); (iv) Map the structural plasticity for the cis cases of the X-Pro-Phe sequence motif (Table 2). Clustering to families was performed according to root mean square deviation fitting.

### Tumour Cell Proliferation Assay

Proliferation was assessed using the MTT assay. Early log phase cells were seeded into micro-titre plates and allowed to grow overnight. AII analogues were then added in serial dilutions. Fresh drug was added every 24 hours. Proliferation was assessed at 24 hour intervals using the MTT assay according to the manufacturer's protocol. IC50 values were calculated as the concentration of agent required to cause a 50% reduction in proliferation relative to untreated controls and/or controls treated with drug vehicle only. Each study was done at least twice and in duplicates of 6.

### Wound Healing Assay

The effect of AII analogues on cellular migration was assessed in wound healing assays which were performed using a scratch protocol. Sub-confluent cells were used (assay done in 35 mm dishes). Cells were grown in low serum medium overnight, then wounded by scratching with a sterile pipette tip using a standard protocols. Cells were exposed to serial dilutions of AII analogues immediately after wounding. The gap between the scratched cell fronts was monitored microscopically.

### REFERENCES

1. Warne, T. et al. Structure of a beta1-adrenergic G-protein-coupled receptor. Nature 454, 486-91 (2008).
2. Fredriksson, R. & Schioth, H. B. The repertoire of G-protein-coupled receptors in fully sequenced genomes. Mol Pharmacol 67, 1414-25 (2005).
3. Mirzadegan, T., Benko, G., Filipek, S. & Palczewski, K. Sequence analyses of G-protein-coupled receptors: similarities to rhodopsin. Biochemistry 42, 2759-67 (2003).
4. Jaakola, V. P. et al. The 2.6 Angstrom Crystal Structure of a Human A2A Adenosine Receptor Bound to an Antagonist. Science (2008).
5. Cherezov, V. et al. High-resolution crystal structure of an engineered human beta2-adrenergic G protein-coupled receptor. Science 318, 1258-65 (2007).
6. Palczewski, K. et al. Crystal structure of rhodopsin: A G protein-coupled receptor. Science 289, 739-45 (2000).
7. Wu, B. et al. Structures of the CXCR4 chemokine GPCR with small-molecule and cyclic peptide antagonists. Science 330, 1066-71.
8. Baker, J. G. The selectivity of beta-adrenoceptor antagonists at the human beta1, beta2 and beta3 adrenoceptors. Br J Pharmacol144, 317-22 (2005).
9. Hubbell, W. L., Altenbach, C., Hubbell, C. M. & Khorana, H. G. Rhodopsin structure, dynamics, and activation: a perspective from crystallography, site-directed spin labeling, sulfhydryl reactivity, and disulfide cross-linking. Adv Protein Chem 63, 243-90 (2003).
10. Van Eps, N., Oldham, W. M., Hamm, H. E. & Hubbell, W. L. Structural and dynamical changes in an alpha-subunit of a heterotrimeric G protein along the activation pathway. Proc Natl Acad Sci USA 103, 16194-9 (2006).
11. Sarkar, P., Reichman, C., Saleh, T., Birge, R. B. & Kalodimos, C. G. Proline cis-trans isomerization controls autoinhibition of a signaling protein. Mol Cell 25, 413-26 (2007).
12. Lu, K. P., Finn, G., Lee, T. H. & Nicholson, L. K. Prolyl cis-trans isomerization as a molecular timer. Nat Chem Biol 3, 619-29 (2007).
13. Nicholson, L. K. & Lu, K. P. Prolyl cis-trans Isomerization as a molecular timer in Crk signaling. Mol Cell 25, 483-5 (2007).
14. Sarkar, P., Saleh, T., Tzeng, S. R., Birge, R. B. & Kalodimos, C. G. Structural basis for regulation of the Crk signaling protein by a proline switch. Nat Chem Biol 7, 51-7.
15. Tzakos, A. G. et al. On the molecular basis of the recognition of angiotensin II (AII). NMR structure of AII in solution compared with the X-ray structure of AII bound to the mAb Fab131. Eur J Biochem 270, 849-60 (2003).
16. Rosenstrom, U. et al. Synthesis and AT2 receptor-binding properties of angiotensin II analogues. J Pept Res 64, 194-201 (2004).
17. Kaschina, E. & Unger, T. Angiotensin AT1/AT2 receptors: regulation, signaling and function. Blood Press 12, 70-88 (2003).
18. de Gasparo, M., Catt, K. J., Inagami, T., Wright, J. W. & Unger, T. International union of pharmacology. XXIII. The angiotensin II receptors. Pharmacol Rev 52, 415-72 (2000).
19. Ichiki, T. et al. Effects on blood pressure and exploratory behaviour of mice lacking angiotensin II type-2 receptor. Nature 377, 748-50 (1995).
20. Doi, C. et al. Angiotensin II type 2 receptor signaling significantly attenuates growth of murine pancreatic carcinoma grafts in syngeneic mice. BMC Cancer 10, 67.
21. Pickel, L. et al. Overexpression of angiotensin II type 2 receptor gene induces cell death in lung adenocarcinoma cells. Cancer Biol Ther 9.
22. Widdop, R. E., Jones, E. S., Hannan, R. E. & Gaspari, T. A. Angiotensin AT2 receptors: cardiovascular hope or hype? Br J Pharmacol 140, 809-24 (2003).
23. Noda, K., Saad, Y. & Karnik, S. S. Interaction of Phe8 of angiotensin II with Lys199 and His256 of AT1 receptor in agonist activation. J Biol Chem 270, 28511-4 (1995).
24. Noda, K. et al. Tetrazole and carboxylate groups of angiotensin receptor antagonists bind to the same subsite by different mechanisms. J Biol Chem 270, 2284-9 (1995).
25. Pulakat, L., Tadessee, A. S., Dittus, J. J. & Gavini, N. Role of Lys215 located in the fifth transmembrane domain of the AT2 receptor in ligand-receptor interaction. Regul Pept 73, 51-7 (1998).
26. Deraet, M. et al. Angiotensin II is bound to both receptors AT1 and AT2, parallel to the transmembrane domains and in an extended form. Can J Physiol Pharmacol 80, 418-25 (2002).
27. Thomas, K. M., Naduthambi, D. & Zondlo, N. J. Electronic control of amide cis-trans isomerism via the aromatic-prolyl interaction. J Am Chem Soc 128, 2216-7 (2006).
28. Morris, K. F. & Johnson, C. S. J. Diffusion-ordered two-dimensional nuclear magnetic resonance spectroscopy. J. Am. Chem. Soc. 114, 3139-3141 (1992).
29. Johnson, C. Diffusion ordered nuclear magnetic resonance spectroscopy: principles and applications. PROGRESS IN NUCLEAR MAGNETIC RESONANCE SPECTROSCOPY 34, 203-256 (1999).
30. Costa-Neto, C. M. et al. Mutational analysis of the interaction of the N- and C-terminal ends of angiotensin II with the rat AT(1A) receptor. Br J Pharmacol 130, 1263-8 (2000).
31. Boucard, A. A. et al. Photolabeling identifies position 172 of the human AT(1) receptor as a ligand contact point: receptor-bound angiotensin II adopts an extended structure. Biochemistry 39, 9662-70 (2000).
32. Pulakat, L., Mandavia, C. H. & Gavini, N. Role of Phe308 in the seventh transmembrane domain of the AT2 receptor in ligand binding and signaling. Biochem Biophys Res Commun 319, 1138-43 (2004).
33. Kurfis, J., Knowle, D. & Pulakat, L. Role of Arg182 in the second extracellular loop of angiotensin II receptor AT2 in ligand binding. Biochem Biophys Res Commun 263, 816-9 (1999).
34. Costanzi, S. On the applicability of GPCR homology models to computer-aided drug discovery: a comparison between in silico and crystal structures of the beta2-adrenergic receptor. J Med Chem 51, 2907-14 (2008).
35. Tamura, M. et al. Lipopolysaccharides and cytokines downregulate the angiotensin II type 2 receptor in rat cardiac fibroblasts. Eur J Pharmacol 386, 289-95 (1999).
36. Meffert, S., Stoll, M., Steckelings, U. M., Bottari, S. P. & Unger, T. The angiotensin II AT2 receptor inhibits proliferation and promotes differentiation in PC12W cells. Mol Cell Endocrinol 122, 59-67 (1996).
37. Mehta, P. K. & Griendling, K. K. Angiotensin II cell signaling: physiological and pathological effects in the cardiovascular system. Am J Physiol Cell Physiol 292, C82-97 (2007).
38. Li, J. M. et al. Angiotensin II-induced neural differentiation via angiotensin II type 2 (AT2) receptor-MMS2 cascade involving interaction between AT2 receptor-interacting protein and Src homology 2 domain-containing protein-tyrosine phosphatase 1. Mol Endocrinol 21, 499-511 (2007).
39. Cao, Z. et al. Angiotensin type 2 receptor is expressed in the adult rat kidney and promotes cellular proliferation and apoptosis. Kidney Int 58, 2437-51 (2000).
40. Rompe, F., Unger, T. & Steckelings, U. M. The angiotensin AT2 receptor in inflammation. Drug News Perspect 23, 104-11.
41. Steckelings, U. M. et al. Non-peptide AT2-receptor agonists. Curr Opin Pharmacol.
42. Halab, L. & Lubell, W. D. Effect of sequence on peptide geometry in 5-tert-butylprolyl type VI beta-turn mimics. J Am Chem Soc 124, 2474-84 (2002).
43. Hunyady, L., Baukal, A. J., Balla, T. & Catt, K. J. Independence of type I angiotensin II receptor endocytosis from G protein coupling and signal transduction. J Biol Chem 269, 24798-804 (1994).
44. Magnani, F., Shibata, Y., Serrano-Vega, M. J. & Tate, C. G. Co-evolving stability and conformational homogeneity of the human adenosine A2a receptor. Proc Natl Acad Sci USA 105, 10744-9 (2008).
45. Schaffner, W. & Weissmann, C. A rapid, sensitive, and specific method for the determination of protein in dilute solution. Anal Biochem 56, 502-14 (1973).
46. Tamura, M., Wanaka, Y., Landon, E. J. & Inagami, T. Intracellular sodium modulates the expression of angiotensin II subtype 2 receptor in PC12W cells. Hypertension 33, 626-32 (1999).
47. Andersen, N. H., Neidigh, J. W., Harris, S. M., Lee, G. M., Liu, Z. H. & Tong, H. Extracting Information from the Temperature Gradients of Polypeptide NH Chemical Shifts. 1. The Importance of Conformational Averaging. J. Am. Chem. Soc. 119, 8547-8561 (1997).
48. Wishart, D. S., Bigam, C. G., Holm, A., Hodges, R. S. & Sykes, B. D. 1H, 13C and 15N random coil NMR chemical shifts of the common amino acids. I. Investigations of nearest-neighbor effects. J Biomol NMR 5, 67-81 (1995).
49. Wang, G. & Dunbrack, R. L., Jr. PISCES: a protein sequence culling server. Bioinformatics 19, 1589-91 (2003).

Unless otherwise stated, any methods and materials similar or equivalent to those described herein can be used in the practice or testing of the present invention. Methods, devices, and materials suitable for such uses are described above. All publications cited herein are incorporated herein by reference in their entirety for the purpose of describing and disclosing the methodologies, reagents, and tools reported in the publications that might be used in connection with the invention.

1. A process for preparing a selective ligand for an angiotensin II receptor comprising the steps of:
(i) selecting a motif of a ligand for said receptor which is susceptible to cis-trans isomerisation;
(ii) comparing said ligand with sequences in a database comprising said motif and determining the influence of substitutions in or close to said motif on the formation of a cis or trans isomer;
(iii) effecting a substitution in said ligand in accordance with said comparison in step (ii), thus favouring a cis or trans isomer in said ligand.

2. A process according to claim 1, wherein said motif comprises proline.

3. A process according to claim 2, wherein said motif comprises X₁-Pro-X₂, wherein X₁ and X₂ are the same or different and can be any amino acid.

4. A process according to claim 3, wherein X₂ is Phe.

5. A process according to claim 1, wherein said ligand is selective for AT2R.

6. A process according to claim 1, wherein said ligand is selective for AT1R.

7. A process according to claim 5, wherein said ligand is selective for AT2R and displays increased cis-isomerisation in solution.

8. A process according to claim 1, wherein said ligand is a mutant of Angiotensin I, II, III or IV, or saralasin.

9. A process according to claim 5, wherein said motif is His-Pro-Phe or His⁶-Pro⁷-Phe⁸.

10. A process according to claim 9, wherein said His or said His⁶ residue is replaced with Tyr, creating a Tyr-Pro-Phe motif, and a Tyr⁶ analogue of Angiotensin II (AII).

11. A process according to claim 9, wherein a 4-substituted Phe residue in which an electron-donating or an electron-withdrawing group is introduced to substitute the hydrogen in the para-position of the phenylalanine ring.

12. A ligand for the Angiotensin II receptor comprising:
i) the sequence Tyr-Pro-Phe;
ii) the sequence Asp-Arg-Val-Tyr-Ile-Tyr-Pro-Phe;
iii) a 4-substituted Phe residue in which an electron-donating or an electron-withdrawing group is present said ligand in lieu of the hydrogen in the para-position of the phenylalanine ring;
iv) the sequence Asp-Arg-Val-Tyr-Ile-Phe-Pro-Phe wherein Phe⁶ is substituted at the 4 position; or
v) a mutant of Angiotensin II, in which the formation of one or more cis or trans isomers is favoured in comparison to wild-type Angiotensin II.

13. A pharmaceutical composition comprising a ligand selected according to claim 1.

14. A pharmaceutical composition comprising a ligand according to claim 12 and a pharmaceutically acceptable carrier.

15. A method of inhibiting the growth of pancreatic carcinoma cells or lung adenocarcinoma cells in a patient through AT2R signaling, comprising administering to said patient a ligand according to claim 13 or claim 14.

16. A pharmaceutical composition according to claim 13 or 14 and an AT1 antagonist, and wherein optionally said AT1 antagonist is present in said pharmaceutical composition.

17. A method for treating a tumour in a patient in need of tumour therapy, comprising administering to said patient a pharmaceutically effective amount of a pharmaceutical composition according to claim 13 or 14.

18. A method for treating a tumour in a patient in need of spinal cord therapy, comprising administering to said patient a pharmaceutically effective amount of a ligand according to claim 1 or claim 12.

19. The method according to claim 17, wherein said ligand comprises [Y]⁶-AII ligand.

20. The method according to claim 18, wherein said ligand comprises [Y]⁶-AII ligand.
